(19) United States
(12) Patent Application Publication
(10) Pub. No.: US 2005/0214220 A1
(43) Pub. Date: Sep. 29, 2005

(54) Tumor model

(75) Inventor: Ralf Brandt, Adelaide (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: vivoPharm Pty Ltd., Adelaide (AU)

(21) Appl. No.: 11/089,545

(22) Filed: Mar. 23, 2005

(30) Foreign Application Priority Data
Mar. 24, 2004 (AU) 2004901532

(51) Int. Cl. A01K 67/027; A61K 49/00

(52) U.S. Cl. 424/9.2; 800/10; 800/18

(57) ABSTRACT

The present invention relates a method of forming an orthotopic solid tumor in a host. The method includes the step of introducing transformed cells into an orthotopic site in the host and allowing the introduced cells to form a tumor. The cells introduced into the orthotopic site are transformed by the introduction of exogenous nucleic acid into the cells and host tissue is not removed from the orthotopic site prior to the introduction of the transformed cells into the orthotopic site.

### CROSS-REFERENCE TO RELATED APPLICATION

This application claims priority to Australian Patent Application No. 2004901532 filed Mar. 24, 2004, which application is incorporated herein by reference in its entirety.

### BACKGROUND OF THE INVENTION

1. Field of the Invention

The present invention relates to a method of forming a tumor in a host, and to a method of determining the efficacy of a compound as an anti-tumor agent.

The present invention also relates to a method of identifying an anti-tumor agent, and to methods of identifying oncogenic nucleic acids and identifying nucleic acids that regulate tumor development.

2. Description of the Related Art

There is a continuing need for the identification of new agents with anti-tumor properties. However, a major obstacle to the identification of such agents is the lack of suitable animal tumor models. In particular, existing animal tumor models are generally not amenable to the generation of the large number of animals required for rigorous testing of the efficacy of a potential anti-tumor agent. In addition, many existing animal tumor models produce tumors that do not adequately reflect the characteristics of tumors normally found in humans.

For example, human tumor xenografts in immunodeficient mice generally cannot provide the large number of animals required for rigorous testing of an anti-tumor agent. In addition, depending upon the site of implantation of the xenograft, the tissue architecture surrounding the xenograft may also not reflect the natural environment in which the tumor normally develops.

In transgenic animal tumor models, in which an animal either expresses an oncogene or carries one or more mutations that result in spontaneous tumor growth, these models generally have the disadvantage of a significant latent period before a tumor develops.

Animal tumor models may also be used to determine whether a particular gene has oncogenic potential, and if so, what are the particular characteristics of cells transformed by such an oncogene. Generally, transgenic or knock-out mice are used as the animal model in such situations. However, because the process of cell transformation may often require multiple genetic changes, the generation of transgenic or knock-out mice with the requisite changes may not be possible or readily achievable. For the same reason, transgenic or knock-out animal models are also not particularly useful for screening the effects of different mutations in genes on cell transformation and tumor phenotype.

Therefore there is a need to identify new animal tumor models that can be used to test the efficacy of anti-tumor agents, and in particular, to test the efficacy of anti-tumor agents on tumor models that more closely reflect the characteristics of tumors normally found in vivo. There is also a need to identify new animal models that allow the oncogenic effects of genes to be tested in an environment that more adequately reflects the normal environment in which tumors develop.

The present invention relates to the development of an orthotopic animal tumor model that can be used to quickly and reliably test the efficacy of new anti-tumor agents. The animal tumor model can also be used to identify oncogenic genes and to determine the effects of a particular gene on the development of tumors.

Throughout this specification reference may be made to documents for the purpose of describing various aspects of the invention. However, no admission is made that any reference cited in this specification constitutes prior art. In particular, it will be understood that the reference to any document herein does not constitute an admission that any of these documents forms part of the common general knowledge in the art in any country. The discussion of the references states what their authors assert, and the applicant reserves the right to challenge the accuracy and pertinency of any of the documents cited herein.

### BRIEF SUMMARY OF THE INVENTION

The present invention provides in certain embodiments a method of forming an orthotopic solid tumor in a host, the method including the step of introducing cells into an orthotopic site in the host and allowing the introduced cells to form a tumor, the cells being transformed by the introduction of exogenous nucleic acid into the cells, wherein host tissue is not removed from the orthotopic site prior to the introduction of the cells.

The present invention also provides in certain embodiments an orthotopic solid tumor, the orthotopic solid tumor being produced by introducing cells into an orthotopic site in a host and allowing the introduced cells to form a tumor, the cells being transformed by the introduction of exogenous nucleic acid into the cells, wherein host tissue is not removed from the orthotopic site prior to the introduction of the cells.

The present invention also provides in certain embodiments an animal including an orthotopic solid tumor, the orthotopic solid tumor being produced by introducing cells into an orthotopic site in an animal and allowing the introduced cells to form a tumor, the cells being transformed by the introduction of exogenous nucleic acid into the cells, wherein host tissue is not removed from the orthotopic site prior to the introduction of the cells.

The present invention also provides in certain embodiments a method of determining the efficacy of a compound as an anti-tumor agent, the method including the steps of:
(a) producing an orthotopic solid tumor in a host by introducing cells into an orthotopic site in the host and allowing the cells to form a tumor, the cells being transformed by the introduction of exogenous nucleic acid into the cells, wherein host tissue is not removed from the orthotopic site prior to the introduction of the cells;
(b) administering an amount of a compound to the host; and
(c) determining the efficacy of the compound as an anti-tumor agent by the effect of the compound on the tumor.

The present invention also provides in another embodiment a method of identifying an anti-tumor agent, the method including the steps of:
(a) producing an orthotopic solid tumor in a host by introducing cells into an orthotopic site in the host and allowing the cells to form a tumor, the cells being transformed by the introduction of exogenous nucleic acid into the cells, wherein host tissue is not removed from the orthotopic site prior to the introduction of the cells;
(b) administering an amount of a compound to the host; and
(c) identifying the compound as an anti-tumor agent by the ability of the compound to inhibit development of the tumor.

The present invention also provides in another embodiment a method of identifying an oncogenic nucleic acid, the method including the steps of:
(a) introducing a nucleic acid into a cell;
(b) introducing cells with the introduced nucleic acid into an orthotopic site in a host, wherein host tissue is not removed from the orthotopic site prior to the introduction of the cells;
(c) determining the ability of the cells to form a tumor; and
(d) identifying the nucleic acid as an oncogenic nucleic acid by the ability of the cells to form a tumor in the host.

The present invention also provides in certain embodiments a method of identifying a nucleic acid that regulates tumor development, the method including the steps of:
(a) introducing a nucleic acid into a cell;
(b) introducing cells with the introduced nucleic acid into an orthotopic site in a host and allowing the cells to form a tumor, wherein host tissue is not removed from the orthotopic site prior to the introduction of the cells; and
(c) identifying the nucleic acid as a nucleic acid that regulates tumor development by the ability of the nucleic acid to regulate the development of the tumor formed.

The present invention also provides in certain embodiments a method of propagating a solid tumor, the method including the steps of:
(a) forming an orthotopic tumor in a host by introducing cells into an orthotopic site in a host and allowing the introduced cells to form a tumor, the cells being transformed by the introduction of exogenous nucleic acid into the cells, wherein host tissue is not removed from the orthotopic site prior to the introduction of the cells;
(b) removing a tumor fragment from the host;
(c) introducing the tumor fragment into an orthotopic site in a new host, wherein host tissue is not removed from the orthotopic site prior to introduction of the tumor fragment; and
(d) forming a tumor in the new host from the tumor fragment.

Certain embodiments of the present invention arise out of studies into orthotopic animal tumor models. In particular, it has been found that in the case of cells transformed by the introduction of an oncogenic nucleic acid, the introduction of the transformed cells into an orthotopic site that has not been cleared of host tissue results in tumors with characteristics that better reflect the characteristics of clinical tumors. The tumors so produced are more reliable indicators as to the efficacy of potential anti-tumor agents. In addition, this method allows the determination of the effects that a particular gene or mutation thereof may have on tumorigenesis or tumor development in vivo.

Various terms that will be used throughout the specification have meanings that will be well understood by a skilled addressee. However, for ease of reference, some of these terms will now be defined.

The term “orthotopic tumor” as used throughout the specification is to be understood to mean any tumor formed in a particular organ in a host in which the cells in the tumor are derived from the same or similar cell type as that of the cells of the organ in which the tumor is present. For example, transformed cells from a mammary epithelial cell line may be introduced into mammary tissue in a host.

In this regard, the term “orthotopic site” is to be understood to mean a site in a host that has cells of the same or similar cell type as the cells being introduced.

The term “transformed” as used throughout the specification in relation to cells is to be understood to mean a cell that is immortalized and whose growth is not contact inhibited by other cells. A cell that is transformed may also no longer show a dependence on exogenous growth factors and/or anchorage dependent growth. A number of assays for cell transformation are known in the art, including focus forming assays and anchorage independent growth assays.

The term “nucleic acid” as used throughout the specification is to be understood to mean any polynucleotide or oligonucleotide. The nucleic acid may be DNA or RNA and may be single stranded or double stranded. The nucleic acid may be any type of nucleic acid, including a nucleic acid that is an oncogene or a potential oncogene, a nucleic acid of viral origin, a nucleic acid of genomic origin, a nucleic acid of cDNA origin (i.e., derived from a mRNA), or a nucleic acid of synthetic origin.

The term “exogenous nucleic acid” as used throughout the specification is to be understood to mean any nucleic acid that is introduced into a cell. Examples of exogenous nucleic acids include viral DNA or RNA introduced into a cell by infection of transfection; vector DNA expressing a cloned insert introduced into a cell by transient or stable transfection; or any double stranded or single stranded DNA or RNA introduced into a cell including oligonucleotides, siRNA, antisense nucleic acids, aptamers, or ribozymes.

In the context of the present invention, it will be appreciated that the exogenous nucleic acid may be introduced into cells before or after the cells have been introduced into the orthotopic site.

The term “oncogenic” as used throughout the specification is to be understood to mean any nucleic acid that has the capacity to transform a cell. In this regard, a proto-oncogene will be understood to mean a nucleic acid that normally does not have the capacity to transform a cell, but when mutated has the ability to transform a cell.

The term “anti-tumor agent” as used throughout the specification is to be understood to mean any compound that is able to inhibit tumor development including inhibition of tumor establishment, and/or inhibition of tumor growth, including inhibition of tumor establishment and/or growth by the effect of the compound on factors produced by the host. For example, an anti-tumor agent may act directly on tumor cells, the anti-tumor agent may modulate the secretion of autocrine and paracrine factors produced by tumor cells, the anti-tumor agent may inhibit angiogenesis of the tumor, or the anti-tumor agent may modulate the secretion of paracrine factors produced by the host that affect tumor development.

The term “inhibit” as used throughout the specification is to be understood to mean a reduction in the progress of a process, including the start, continuation or termination of a process. For example, inhibition of tumor development may result from a reduction in the rate of growth of a tumor, and/or may result.

As mentioned above, in one embodiment the present invention provides a method of forming an orthotopic solid tumor in a host, the method including the step of introducing cells into an orthotopic site in the host and allowing the introduced cells to form a tumor, the cells being transformed by the introduction of exogenous nucleic acid into the cells, wherein host tissue is not removed from the orthotopic site prior to the introduction of the cells.

This embodiment of the present invention is useful, for example, in the production of tumors in animals than can be used to determine the efficacy of potential anti-tumor agents.

The orthotopic solid tumor in the various embodiments of the present invention may be a solid tumor in any organ of the host. For example, the orthotopic solid tumor may be a tumor in the bladder, bone, bowel, brain, breast or mammary tissue, cervix, colon, eye, intestines, kidney, larynx, liver, lung, muscle, mouth, esophagus, ovaries, pancreas, prostate, skin, stomach, testicles, thyroid cancer, and uterus.

In the case of an orthotopic tumor in breast or mammary tissue of a mouse, preferably the tumor is in the mammary gland fat pad.

The present invention also contemplates, according to certain embodiments, solid tumors produced by the methods of the present invention.

Accordingly, in another embodiment the present invention provides an orthotopic solid tumor, the orthotopic solid tumor produced by introducing cells into an orthotopic site in a host and allowing the introduced cells to form a tumor (or tumor fragments), the cells being transformed by the introduction of exogenous nucleic acid into the cells, wherein host tissue is not removed from the orthotopic site prior to the introduction of the cells.

The host for development of the orthotopic solid tumor in the various embodiments of the present invention may be any suitable animal host.

In this regard, the term “animal” is to be understood to mean any non-human animal, including for example mammals, birds, reptiles, amphibians, and fish. Preferably, the non-human animal is in certain embodiments a mammal (for example rodent, mouse, rat, rabbit, monkey, dog, cat, and pig). It will also be appreciated that the animal may be a transgenic animal. More preferably, the animal is a rodent.

Preferably the animal host is a mouse or rat.

The host may be an immune competent host or an immunodeficient host. In mice, examples of immunodeficient mice include nude mice, scid mice or rag1 mice.

Accordingly, in another embodiment the present invention also provides an animal including an orthotopic solid tumor, the orthotopic solid tumor being produced by introducing cells into an orthotopic site in an animal and allowing the introduced cells to form a tumor, the cells being transformed by the introduction of exogenous nucleic acid into the cells, wherein host tissue is not removed from the orthotopic site prior to the introduction of the cells.

The cells for introduction into the orthotopic site may be cells that have been transformed by the introduction of an exogenous nucleic acid (such as a nucleic acid encoding an oncogene), or alternatively may be cells that are to be transformed by an exogenous nucleic acid after the cells have been introduced into the orthotopic site. The cells may be of any specific lineage, so long as the lineage of the cells is related to the cells at their site of introduction. In this regard, the cells will not form a tumor unless transfected with a transforming exogenous nucleic acid.

The cells may be cells derived from cultured cell lines, or alternatively, be cells derived from primary cells. Preferably according to certain embodiments, the cells are cells derived from a cultured cell line, which are then transformed by an exogenous nucleic acid.

In the case of cells derived from cultured cell lines, the cells for introduction into the host will be transformed with an exogenous nucleic acid.

In the case of cells derived from primary cells, the cells may be primary cells, cells derived from primary cells, or primary cells cultured in vitro. Primary cells may be transformed with an exogenous nucleic acid prior to or after their isolation, or alternatively may be transformed with an exogenous nucleic acid after the cells have been introduced into the orthotopic site.

For the development of orthotopic mammary tumors in mice, a suitable cell line for introduction is the HC11 cell line, which is then transformed by exogenous nucleic acid. HC11 is a non-transformed normal mammary epithelial cell line, as described in Ball et al. (1988) EMBO J. 7(7):2089-2095.

The cells may be derived from the same species as the host, or alternatively, may be derived from a different species. In the case of cells for introduction into a host of the same species, the host is preferably an immunocompetent host. In the case of cells for introduction of cells into a host of a different species, preferably the host is an immunodeficient host.

In contrast to the surgical removal of tissue from the orthotopic site prior to introduction of the cells, the orthotopic tumors of the present invention involve the introduction of cells into an orthotopic site that has not had host tissue removed from the site.

Without being bound by theory, it appears that the introduction of cells into an orthotopic site that has not had host tissue removed results in an environment for tumor formation that more adequately represents the environment in which tumors normally develop, due to the natural paracrine interactions between host and tumor tissue.

Tumors so formed are closer to the clinical situation and therefore better models for testing the efficacy of anti-tumor agents.

To introduce the cells into an orthotopic site that has not been cleared of epithelial tissue, the cells are introduced by direct implantation into the site. Methods of direct implantation include inoculation of cells into the orthotopic site, injection of cells into the orthotopic site, or alternatively, by separation of host tissue by surgical means followed by implantation and suturing. Preferably, the cells are introduced into the orthotopic site by inoculation or injection.

The number of cells introduced into the orthotopic site is not particularly limited, and will depend upon factors such as the desired latency period for tumor development.

Preferably according to certain embodiments, the number of cells introduced into the orthotopic site is in the range from 5×10⁵ to 3×10⁷ cells. Most preferably, the number of cells introduced into the orthotopic site is in the range from 0.5-1.0×10⁶ cells.

The cells may be introduced into the orthotopic site by a suitable method known in the art. For example, the cells may be introduced by injection of a suspension of cells directly into the orthotopic site. In this case, if the cells are adherent cells grown in culture, the cells may be trypsinized, washed and resuspended in a suitable medium for introduction into the host, such as PBS or HBSS. If the cells are non-adherent cells grown in culture, the cells may be pelleted by centrifugation, washed and resuspended in a suitable medium, such as PBS or HBSS.

The introduction of a suspension of cells by injection into the orthotopic site is preferred in certain embodiments.

For example, in the case of introducing cells into the prostate, a suspension of cells can be introduced directly into the ventral lobe. No suturing is required in this case.

If the cells for inoculation are derived from a tumor biopsy (either previously transformed with an exogenous nucleic acid, or to be transformed with an exogenous nucleic acid before or after introduction into the orthotopic site), the cells present in the tumor may be disaggregated by a suitable method known in the art and the cells injected into the orthotopic site. The cells present in the tumor may also be disaggregated and then cultured in vitro, before resuspension and introduction into the orthotopic site.

Alternatively, the tumor can be cut into 1-2 mm³ fragments (explants) and these explants introduced into the host tissue, for example by using a trocan needle.

In the case of a tumor sample being disaggregated and introduced as a suspension, an example of a suitable method for the introduction of a lung carcinoma may be as follows: a fresh surgically-resected sample of lung carcinoma is processed under sterile conditions. Viable tumor specimens of approximately 1 cm³ in size may then be minced finely with a scalpel. The minced tumor tissue may then be disaggregated into a single-cell suspension using an enzyme mixture consisting of 0.5 mg/ml collagenase, 0.2 mg/ml hyaluronidase and 0.2 mg/ml deoxyribonuclease in serum-free RPMI-1640 (Sigma Chemical Co., St. Louis, USA). After washing twice with RPMI-1640 supplemented with 10% heart-inactivated fetal bovine serum (Biosciences Pty Ltd., Australia), the single cells and small clusters may be seeded in 25 cm² flasks coated with collagen type I (Asahi Techno Grass, Tokyo, Japan) containing RPMI-1640 medium with 10% heart-inactivated fetal bovine serum and cultured at 37° C. in a humidified incubator with 5% CO₂ in air. The cells are harvested for implantation at 70 to 80% confluence using 0.05% trypsin (Gibco BRL, NY, USA) and 1 mmol/l ethylenediamine-tetraacetic acid (Wako Pure Chemical Industries Ltd., Osaka, Japan) in phosphate-buffered saline (Nissui Pharmaceutical Co. Ltd., Tokyo, Japan). These cells are then washed in RPMI-1640 medium and resuspended to final concentration of 1×10⁶ cells/ml in RPMI-1640 medium containing 0.1% bovine serum albumin (Boehringer Mannheim, Germany). The cells may then be injected into the orthotopic site.

Alternatively, as discussed above, the cells may be introduced into the orthotopic site as a mass of cells (eg an explant), such as derived from a surgical sample. Once again, host tissue is not removed from the orthotopic site prior introduction of the host cells.

For surgical introduction of orthotopic samples into mice, the implantation should generally take place within 24 hours of surgical excision. Before transplantation, each specimen for introduction should be preferably inspected, and all necrotic and suspected necrotic tumor tissue removed. To take into account tumor heterogeneity, each specimen should also be equally divided into a number of parts, separated and each part subsequently cut into small pieces. A size of about 1-2 mm³ size is suitable for implantation in mice.

In the case of orthotopic introduction of a prostate cancer sample into mice, an example of a suitable method for implantation may be as follows: the mice are first anaesthetized by isoflurane inhalation. Tumor fragments may then be prepared and implanted in the dorsolateral lobe of the prostate. After proper exposure of the bladder and prostate following a lower midline abdominal incision, the capsule of the prostate may then be opened after injection of sterile air (syringe with 2 μM filter) and the tumor fragments inserted into the capsule. The capsule may then be then closed with an 8-0 surgical suture. The incision in the abdominal wall is then closed with a 6-0 surgical suture in one layer.

In the case of orthotopic introduction of lung cancer tumors into mice, an example of a suitable method for the introduction may be as follows: the mice are first anaesthetized by isoflurane inhalation. The animals are then put in a position of right lateral decubitus, with four limbs restrained. A 0.8 cm transverse incision of skin may then be made in the left chest wall. Chest muscles are separated by sharp dissection and costal and intercostal muscles are exposed. A 0.4-0.5 cm intercostal incision between the third and fourth rib on the chest wall may then be made and the chest wall opened. The left lung is taken up with a forceps and tumor fragments sewn promptly into the upper lung with one suture. The lung is then returned into the chest cavity. The incision in the chest wall is then closed by a 6-0 surgical suture. The closed condition of the chest wall is examined immediately and if a leak exists, it is to be closed by additional sutures. After closing the chest wall, an intrathoracic puncture is made by using a 3-ml syringe and 30 G needle to withdraw the remaining air in the chest cavity. After the withdrawal of air, a completely inflated lung will be seen through the thin chest wall of the mouse. The skin and chest muscle may then be closed with a 6-0 surgical suture in one layer.

In light of the above, it will also be appreciated that the passaging of tumor fragments is included within the scope of the invention. Thus, tumor fragments from a tumor of the same type may be passaged in the host animal.

Accordingly, in another embodiment the present invention also provides a method of propagating a solid tumor, the method including the steps of:
(a) forming an orthotopic tumor in a host by introducing cells into an orthotopic site in a host and allowing the introduced cells to form a tumor, the cells being transformed by the introduction of exogenous nucleic acid into the cells, wherein host tissue is not removed from the orthotopic site prior to the introduction of the cells;
(b) removing a tumor fragment from the host;
(c) introducing the tumor fragment into an orthotopic site in a new host, wherein host tissue is not removed from the orthotopic site prior to introduction of the tumor fragment; and
(d) forming a tumor in the new host from the tumor fragment.

The present invention also contemplates solid tumors propagated by the above method.

As discussed previously, the cells for introduction into the orthotopic site in certain embodiments of the present invention are cells transformed by the introduction of an exogenous nucleic acid into the cells.

The introduction of an exogenous nucleic acid to transform a cell in certain of the various embodiments of the present invention may be by a suitable method known in the art. In this regard, cell transformation can occur by a number of different mechanisms. For example, cell transformation can occur by infection of a cell with a transforming virus, the introduction of an oncogene or mutated proto-oncogene into the cell, or by the loss of function of a tumor suppressor gene. Methods of mammalian transformation are as generally as described in Aubin, R. (2002) “Mammalian Cell Transformation Protocols” Humana Press.

Preferably, the exogenous nucleic acid is oncogene or proto-oncogene. More preferably, the oncogene or proto-oncogene is an activated oncogene.

In the case of cell transformation by way of infection with a transforming virus, the transforming virus may be a DNA virus or a RNA virus. This transformation represents an example in which the exogenous nucleic acid is introduced into the cell by way of viral infection.

Transforming RNA viruses include (i) acutely transforming or transducing viruses such as Rous Sarcoma Virus; (ii) replication competent viruses that transform by insertional mutagenesis, such as avian leukosis viruses; and (iii) replication competent viruses with trans-acting functions, such as HTLV-1. Transforming DNA viruses include Papovaviruses, Papillomaviruses, Herpesviruses and Hepatitis B virus.

In the case of cell transformation by way of introduction of an oncogene, the oncogene may be introduced into the cell by a suitable method known in the art. For example, the oncogene may be introduced into the cell by viral infection (e.g., as part of a retrovirus), or by way of transient or stable transfection.

Preferably, the introduction of exogenous nucleic acid to transform the cells that are introduced into the orthotopic site occurs prior to the introduction of the cells into the animal.

Accordingly, in a preferred embodiment the present invention provides a method of forming an orthotopic solid tumor in a host, the method including the step of introducing transformed cells into an orthotopic site in the host and allowing the introduced transformed cells to form a tumor, the cells being transformed by the introduction of exogenous nucleic acid into the cells, wherein host tissue is not removed from the orthotopic site prior to the introduction of the transformed cells.

However, it will be appreciated that transformation of the cells (for example by way of a transforming virus) after the cells have been introduced into the host is also included within the scope of the present invention. In this case, cells introduced into the orthotopic site may, for example, be exposed to a transforming virus in situ. For example, a transforming virus may be exposed to the introduced cells in an animal host by directly injecting the virus into the orthotopic site. Alternatively, the animal as a whole may be infected with the transforming viurs.

A number of oncogenes have been identified through the characterization of transforming viruses, by examination of the breakpoints resulting from chromosomal translocation, or by expression cloning of DNA molecules using mesenchymal cells such as NIH3T3. Many oncogenes are strongly homologous to cellular proto-oncogenes. Examples of such proto-oncogenes include src, myc, abl and ras.

Oncogenes and their proto-oncogenes generally fall into five main categories: (i) secreted growth factors, such as SIS; (ii) cell surface receptors, such as erbB2 and FMS; (iii) components of intracellular signal transduction systems such as RAS and ABL; (iv) nuclear DNA-binding proteins, transcription factors such as MYC and JUN; and (v) cyclins, cyclin-dependent kinases and other components of cell cycle regulation.

Examples of growth factors and their receptors that can lead to cell transformation include v-SIS/PDGFB, the EGFR family of tyrosine kinase receptors, RET, c-met, and VEGFR.

In this regard, the epidermal growth factor receptor (EGFR) is one of a family of tyrosine kinases receptors (other members include HER2, HER3 and HER4) and when activated by ligand binding, this family bind and activate a wide range of intracellular signalling molecules such as phospholipase C, GAP and SRC. For instance, deletions that remove the ligand binding capacity in human EGFR and constitutively phosphorylate the protein occur in a high proportion of breast and ovarian tumors. This example resembles the viral oncogene v-erbB, carried by avian erythroblastosis virus, which encodes a truncated form of EGFR that has lost its ligand binding domain and has constitutive protein tyrosine kinase activity.

RET encodes a tyrosine kinase receptor, the ligand for which is glial cell-line-derive neurotrophic factor (GDNF). Oncogenic forms of RET are constitutively phosphorylated and have autophosphorylation activity. Oncogenic mutations in RET are dominantly acting point mutations.

Components of intracellular signal transduction systems involved in cell transformation include the RAS superfamily, which encode GTP-binding domain (G-proteins) involved in signal transduction.

Examples of nuclear transcription factors that are involved in cell transformation include transcription factors that are directly involved in control of growth. Some, such as ETS, MYB and erbA, bind to specific DNA sequences in their monomeric form, while other, such as FOS, JUN, MYC and REL, only interact with DNA as part of complexes with other proteins.

In the case of cell transformation by loss of function of a tumor suppressor gene, the exogenous nucleic acid introduced into the cell may inhibit the expression or function of the tumor suppressor gene by a suitable method known in the art, such as antisense inhibition, siRNA inhibition, or by the use of a ribozyme. Alternatively, all or part of the exogenous nucleic acid may integrate into the tumor suppressor gene (or a regulatory region thereof). Examples of the loss of a tumor suppressor gene that may lead to cell transformation include p53 and Rb.

In a preferred embodiment of the invention, the transformation of the cells introduced into the orthotopic site is by way of introduction of an exogenous nucleic acid that results in constitutive activation of a tyrosine kinase receptor.

Accordingly, in another embodiment the present invention provides a method of forming an orthotopic solid tumor in a host, the method including the step of introducing cells into an orthotopic site in the host and allowing the introduced cells to form a tumor, the cells being transformed by the introduction of exogenous nucleic acid that results in constitutive activation of a tyrosine kinase receptor in the cells, wherein host tissue is not removed from the orthotopic site prior to the introduction of the cells.

Preferably, according to certain embodiments the tyrosine kinase receptor is a receptor from the epidermal growth factor receptor family.

In this regard, the epidermal growth factor receptor family includes EGFR (also known as ErbB-1[RB1] or HER1) and the closely related tyrosine kinase receptors ErbB2 (HER2), ErbB3(HER3) and ErbB4 (HER4).

A number of assays for cell transformation are known in the art. For example, in a focus forming assay, transformation is detected as foci of dense morphologically altered cells in cell monolayers. This assay depends on the loss of contact inhibition following transformation.

In an anchorage independent growth assay, the capacity of cells to grow in the absence of attachment to a solid surface is detected. Primary fibroblasts and many fibroblastic lines must attach to a solid surface before they can divide. Transformed cells do not show this requirement.

Transformed cells will also often show a reduced serum requirement. Thus, often transformed cells may be identified by their ability to grown in medium containing a reduced level of serum.

Generally, exogenous DNA can be introduced into cell by a variety of methods known in the art. A preferred method of introducing exogenous nucleic acid into a cell is by way of retroviral infection. For example, in the case of producing orthotopic mammary tumors in mice, a mammary epithelial cell line may be infected by a retrovirus carrying NeuT, the rat homologue of erbB-2. Cells so transformed may then be introduced into the mammary fat pad of mice to form tumors.

Other methods of introducing exogenous nucleic acid into cells include transformation using calcium phosphate, electroporation, lipofection, and particle bombardment. Methods for introducing exogenous nucleic acids into cells are essentially as described in Sambrook, J, Fritsch, E. F. and Maniatis, T. Molecular Cloning: A Laboratory Manual 2d ed. Cold Spring Harbor Laboratory Press, New York. (1989).

In the case where the exogenous nucleic acid is introduced into cells cloned (or expressed from) a vector, suitable vectors include plasmid vectors and viral vectors.

The vector may also further include regulatory elements for the expression of inserted nucleic acids, for example promoters for driving the expression of an inserted nucleic acid in a particular cell type, poly A signals for efficient polyadenylation of mRNA transcribed from inserted nucleic acids, or other regulatory elements to control translation, transcription or mRNA stability, all known in the art.

The expression in a cell of a particular nucleic acid may be by a suitable method known in the art. For example, a nucleic acid may be isolated and cloned into a suitable expression vector for use in the cell type of interest by methods known in the art. Methods for the isolation of nucleic acid sequences and their cloning into a suitable expression vector are essentially as described in Sambrook, J, Fritsch, E. F. and Maniatis, T. Molecular Cloning: A Laboratory Manual 2d ed. Cold Spring Harbor Laboratory Press, New York. (1989). The recombinant molecule may then be introduced into the cell and the cloned nucleic acid expressed.

In the case of introducing an exogenous nucleic acid into a cell to express a target gene, such as an oncogene, the expression may be achieved by a number of methods known in the art. These include transient or stable transfection of cells with a recombinant nucleic acid encoding the gene of interest under the control of a promoter that is active in the particular cell type.

In the case of introducing an exogenous nucleic acid into a cell to decrease the expression of a target gene, such as a tumor suppressor gene, a decrease in expression may be achieved by a number of methods known in the art. These include the use of an antisense nucleic acid that binds to an endogenous mRNA and which interferes with translation, the use of a molecule that can specifically repress transcription of the endogenous mRNA such as a specific DNA or RNA binding protein, a nucleic acid capable of forming a triple helix structure, a small interfering RNA (si RNA), or a ribozyme that can cleave a specific mRNA.

Generally, the introduction of exogenous nucleic acids to decrease the expression of a target gene will involve constitutive expression in the cell of the nucleic acid. However, under some circumstances it may be appropriate to express the nucleic acid by use of an inducible promoter.

In the case of the use of antisense technology to decrease expression, the antisense nucleic acid will include a sequence complementary to at least a portion of the target RNA. Absolute complementarity, although preferred, is not required, as long as the antisense nucleic acid is capable of hybridizing with the target RNA and thereby interferes with expression from the RNA. As will be appreciated, the ability to hybridize will depend on both the degree of complementarity and the length of the antisense nucleic acid. Methods known in the art may be used to formulate possible antisense nucleic acids.

In the case of using a ribozyme to decrease expression, the functional constraints necessary for a nucleic acid to act as a ribozyme are essentially as described in Haseloff et al., (1988) Nature 334: 585-591; Koizumi et al., (1988) FEBS Lett., 228: 228-230; Koizumi et al., (1988) FEBS Lett., 239: 285-288). Ribozyme methods that involve inducing expression in a cell of ribozyme molecules are essentially as described in Grassi and Marini (1996) Annals of Medicine 28: 499-510; Gibson (1996) Cancer and Metastasis Reviews 15: 287-299.

The mechanism of ribozyme action involves sequence-specific hybridization of the ribozyme molecule to complementary target RNA, followed by endonucleolytic cleavage. Examples of ribozymes that may be used include engineered hammerhead motif ribozyme molecules that can specifically and efficiently catalyze endonucleolytic cleavage of sequences encoding the target sequence.

To design a ribozyme, specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays, as known in the art.

The ribozyme will be expressed in vivo at a sufficient level to be catalytically effective in cleaving mRNA, and thereby modify mRNA abundance in a cell. For example, a ribozyme coding DNA sequence may be synthesized and ligated into a restriction enzyme site in the anticodon stem and loop of a gene encoding a tRNA, which can then be transformed into and expressed in a cell of interest by a method known in the art. Alternately, an inducible promoter can by used so that ribozyme expression can be selectively controlled in a particular cell type.

In the case of using a siRNA to decrease the expression of gene, the functional constraints for the design and expression of the siRNA are essentially as described in Brummelkamp et al. (2002) Science 296(5567):550-553.

It will also be appreciated, that it is possible to reduce expression of a tumor suppressor gene, for example by way of antisense or siRNA technology, alone or in combination with a gain of function as may occur through the introduction of an oncogene.

In a particularly preferred embodiment, the present invention provides a method of forming a mammary tumor in animal, the method including the step of introducing transformed mammary or mammary-derived cells into a site in a mammary gland in the animal and allowing the introduced cells to form a tumor, the cells being transformed by the introduction of an oncogene or proto-oncogene into the cells, wherein animal tissue is not removed from the site prior to the introduction of the transformed cells.

As discussed previously, certain embodiments of the present invention also allow the efficacy of potential anti-tumor agents to be determined.

Accordingly, in another embodiment the present invention provides a method of determining the efficacy of a compound as an anti-tumor agent, the method including the steps of:
(a) producing an orthotopic solid tumor in a host by introducing cells into an orthotopic site in the host and allowing the cells to form a tumor, the cells being transformed by the introduction of exogenous nucleic acid into the cells, wherein host tissue is not removed from the orthotopic site prior to the introduction of the cells;
(b) administering an amount of a compound to the host; and
(c) determining the efficacy of the compound as an anti-tumor agent by the effect of the compound on the tumor.

In this and related embodiments, the orthotopic solid tumor model of the resent invention may be used to test the efficacy of known and candidate anti-tumor agents. For example, in the present the efficacy of a kinase inhibitor of EGF receptor has been determined, as compared to Taxol®, a known anti-tumor agent.

The anti-tumor agent may be a known anti-tumor agent or a candidate anti-tumor agent. In this regard, certain embodiments of the present invention also contemplate the identification of an anti-tumor agent using the method of forming orthotopic solid tumors disclosed herein.

Accordingly, in another embodiment the present invention provides a method of identifying an anti-tumor agent, the method including the steps of:
(a) producing an orthotopic solid tumor in a host by introducing cells into an orthotopic site in the host and allowing the cells to form a tumor, the cells being transformed by the introduction of exogenous nucleic acid into the cells, wherein host tissue is not removed from the orthotopic site prior to the introduction of the cells;
(b) administering an amount of a compound to the host; and
(c) identifying the compound as an anti-tumor agent by the ability of the compound to inhibit development of the tumor.

The present invention also contemplates anti-tumor agents identified by the above method.

Examples of types of anti-tumor agents include small molecules, drugs, antibodies, polypeptides, peptides, enzymes, polysaccharides, glycoproteins, hormones, receptors, ligands, nucleic acids, aptamers, antisense nucleic acids, siRNAs, and ribozymes.

The administration of a test compound (as an anti-tumor agent) may occur at any time and at any desired frequency during formation of the orthotopic tumor, and/or at any time and at any desired frequency after formation of the orthotopic tumor.

For example, for testing the efficacy of an agent on tumor establishment, the agent may administered to the animal at any time before, during and/or after the cells have been introduced into the orthotopic site. Similarly, in the case of testing the efficacy of an agent on tumor growth, the agent may administered to the animal at any time before, during and/or after the cells have been introduced into the orthotopic site.

As will also be appreciated, the efficacy of the compound as an anti-tumor agent will depend on the concentration of the compound delivered to a host subject. Accordingly, the amount of compound administered to a host is not particularly limited.

The compound for testing as an anti-tumor may be administered alone or in the form of a composition. Compositions containing the compound may also contain a preservative, stabiliser, dispersing agent, pH controller or isotonic agent.

The compound for testing as an anti-tumor agent may be prepared into a number of different types of formulation for administration to a host. For example, the compound may be prepared into a variety of preparations in the form of an aqueous solution, an oily preparation, a fatty emulsion, an emulsion, or a gel.

The compound may also be administered containing a pharmaceutically acceptable carrier, diluent, excipient, suspending agent, lubricating agent, adjuvant, vehicle, delivery system, emulsifier, disintegrant, absorbent, preservative, surfactant, colorant, flavourant or sweetener.

For these purposes, the composition may be administered parenterally, orally, or by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the composition will preferably be isotonic with the blood of the host in combination with a pharmaceutically acceptable carrier. Examples of such injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,3-butanediol. Among the acceptable vehicles and solvents that may be employed are water, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

Sterile saline is a preferred carrier. The carrier may also contain minor amounts of additives, such as substances that enhance solubility, isotonicity, and chemical stability, for example anti-oxidants, buffers and preservatives.

If the compound is administered orally, the composition will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

The administration of the compound may utilize controlled release technology. The compound may also be administered as a sustained-release pharmaceutical.

The efficacy of the compound as an anti-tumor agent may be determined by a suitable method known in the art, applicable to the particular tumor model being tested.

Parameters for determining the effect of a compound on tumor development include, for example, proliferation of the tumor, tumor morphology, angiogensis of the tumor, or the rate of formation of metastases. For example, the compound may prevent or inhibit growth of the orthotopic tumor at one or more concentrations.

It will also be appreciated that certain embodiments of the present invention may be used to determine the efficacy of a compound as an anti-metastatic agent.

Accordingly, in another embodiment the present invention provides a method of determining the efficacy of a compound as an anti-metastatic agent, the method including the steps of:
(a) producing an orthotopic solid tumor in a host by introducing cells into an orthotopic site in the host and allowing the cells to form a tumor, the cells being transformed by the introduction of exogenous nucleic acid into the cells, wherein host tissue is not removed from the orthotopic site prior to the introduction of the cells;
(b) administering an amount of a compound to the host; and
(c) determining the efficacy of the compound as an anti-metastatic agent by the ability of the compound to inhibit the development of metastases in the host derived from the orthotopic solid tumor.

Certain embodiments of the present invention also allow the identification of a nucleic acid with oncogenic capacity.

Accordingly, in another embodiment the present invention provides a method of identifying an oncogenic nucleic acid, the method including the steps of:
(a) introducing a nucleic acid into a cell;
(b) introducing cells with the introduced nucleic acid into an orthotopic site in a host, wherein host tissue is not removed from the orthotopic site prior to the introduction of the cells;
(c) determining the ability of the cells to form a tumor; and
(d) identifying the nucleic acid as an oncogenic nucleic acid by the ability of the cells to form a tumor in the host.

Certain related embodiments of the present invention also contemplate oncogenic nucleic acids identified by the above method.

In such embodiments of the present invention, the oncogenic capacity of a articular exogenous nucleic acid to transform a cell may be tested. For example, in the resent study the ability of NeuT (the rat homolgue of erbB-2[RB2]) to function as an oncogenic nucleic acid has been confirmed using the orthotopic tumor model.

The nucleic acid introduced into a cell is any nucleic acid for which the oncogenicity is to be determined.

For example, the nucleic acid may be a candidate oncogene, a nucleic acid that provides a gain of function of a proto-oncogene, or a nucleic acid that functions to inhibit a tumor suppressor gene. The introduction of the nucleic acid into the cell may be by suitable method known in the art. For example, the nucleic acid may be introduced into the cell by viral infection (e.g. as part of a retrovirus), or by way of transient or stable transfection with plasmid constructs including the potentially oncogenic nucleic acid.

Methods for introduction of exogenous nucleic acids into cells are as described previously. As discussed previously, it will be appreciated that the nucleic acid may be introduced into the cells before or after the cells are introduced into the orthotopic site. Preferably, the nucleic acid is introduced into the cells before the cells are introduced into the orthotopic site.

Preferably, the nucleic acid is introduced into a cell by retroviral infection.

It will also be appreciated that the oncogenic nucleic acid may be, for example, a siRNA, an antisense RNA, or a ribozyme.

The formation of an orthotopic tumor in the host after a latent period will indicate that the introduced nucleic acid may function as an oncogenic nucleic acid in the particular cell type selected. The formation of a tumor in the host may be confirmed by a suitable method known in the art.

For example, in the case of identifying whether a nucleic acid may function as an oncogenic nucleic acid in a mammary cell line, a mammary epithelial cell line may be infected by a retrovirus carrying the nucleic acid and the cells with the introduced nucleic acid introduced into the mammary fat pad of mice. The formation of a mammary tumor will indicate that the nucleic acid has oncogenic activity in mammary cells in vivo.

As will be appreciated, these and related embodiments of the present invention also allow the assessment of the oncogenic capacity of a nucleic acid.

Accordingly, in another embodiment the present invention provides a method of assessing the oncogenic capacity of a nucleic acid, the method including the steps of:
(a) introducing a nucleic acid into a cell;
(b) introducing cells with the introduced nucleic acid into an orthotopic site in a host, wherein host tissue is not removed from the orthotopic site prior to the introduction of the cells; and
(c) assessing the oncogenic capacity of the nucleic acid by determining the ability of the cells to form a tumor.

Certain embodiments of the present invention also allow the identification of a nucleic acid that regulates tumor development.

Accordingly, in another embodiment the present invention provides a method of identifying a nucleic acid that regulates tumor development, the method including the steps of:
(a) introducing a nucleic acid into a cell;
(b) introducing cells with the introduced nucleic acid into an orthotopic site in a host and allowing the cells to form a tumor, wherein host tissue is not removed from the orthotopic site prior to the introduction of the cells; and
(c) identifying the nucleic acid as a nucleic acid that regulates tumor development by the ability of the nucleic acid to regulate the development of the tumor formed.

Certain embodiments of the present invention also contemplate nucleic acids identified by the above method.

In these and related embodiments of the present invention, the ability of a particular exogenous nucleic acid to regulate tumor development may be tested.

The nucleic acid introduced into a cell is any nucleic acid for which the ability to regulate tumor development is to be determined.

For example, the nucleic acid may be a candidate oncogene, a nucleic acid that provides a gain of function of a proto-oncogene, a nucleic acid that functions to inhibit a tumor suppressor gene, a nucleic acid that alters tumor angiogenesis, or a nucleic acid that alters tumor phenotype. The introduction of the nucleic acid into the cell may be by suitable method known in the art. For example, the nucleic acid may be introduced into the cell by viral infection (e.g., as part of a retrovirus), or by way of transient or stable transfection with plasmid constructs including the nucleic acid. Methods for introduction of exogenous nucleic acids into cells are as described previously.

Preferably, the nucleic acid is introduced into a cell by retroviral infection.

It will also be appreciated that the nucleic acid may be, for example, a siRNA, an antisense RNA, or a ribozyme.

The effect of the nucleic acid to regulate tumor development may be determined by a suitable method known in the art, and as will be appreciated, will depend upon the particular effect of the nucleic acid on tumor development being determined.

For example, the nucleic acid may have an effect on tumor proliferation. In this case, the growth of the tumor will be altered as compared to control cells without the introduced nucleic acid. Alternatively, the nucleic acid may have an effect on the morphology of the tumor, or angiogenesis of the tumor.

For example, in the case of identifying whether a nucleic acid may function to regulate mammary tumor development, a mammary epithelial cell line may be infected by a retrovirus carrying the nucleic acid and the cells with the introduced nucleic acid introduced into the mammary fat pad of mice. The effect of the nucleic acid on the development of the mammary tumor may then be determined.

As will be appreciated, certain embodiments of the present invention thus also allow the assessment of the capacity of a nucleic acid to regulate tumor development.

Accordingly, in another embodiment the present invention provides a method of assessing the capacity of a nucleic acid to regulate tumor development, the method including the steps of:
(a) introducing a nucleic acid into a cell;
(b) introducing cells with the introduced nucleic acid into an orthotopic site in a host, wherein host tissue is not removed from the orthotopic site prior to the introduction of the cells; and
(c) assessing the capacity of the nucleic acid to regulate tumor development by determining the ability of the cells to regulate development of a tumor formed from the introduced cells.

### DETAILED DESCRIPTION OF THE INVENTION

Reference will now be made to experiments that embody the above general principles of the present invention. However, it is to be understood that the following description is not to limit the generality of the above description.

### Example 1

Cell Lines and Retroviral Infection

HC11 is a mammary epithelial cell line and was grown in RPMI-1640 supplemented with 10% fetal calf serum (FCS), 10 ng/ml EGF (recombinant human, Sigma) and 5 μg/ml insulin (Sigma). BOSC-23 viral packaging cells were propagated in Dulbecco's modified Eagle's medium with 10% FCS. All culture media were supplemented with penicillin and streptomycin.

HC11 cells were infected with the pBabe-puro retrovirus as described in Morgenstern and Land (1990) Nucl. Acids Res. 18:3587-3596. Cells were either infected with the pBabe-puro encoding oncogenic NeuT (pBabe-puro NeuT) or control pBabe-puro. In this case, oncogenic activation is by the ErbB2 receptor tyrosine kinase (RTK). The receptor is constitutively activated in a ligand-independent manner due to spontaneous dimerization.

The retroviral infection of the cells represents the introduction of an exogenous nucleic acid into cells for the purpose of transforming the cells. In this particular case, the introduced exogenous nucleic acid is an oncogenic nucleic acid that transforms the cell by constitutively activating the tyrosine kinase receptor.

High-titer, helper-free recombinant retroviruses were produced from BOSC-23 cells transiently transfected with plasmid DNA, using the CaPO4 technique, as described in Pear et al. (1993) Proc. Natl. Acad. Sci. 90:8392-8396. HC11 cells were infected by exposure to filtered conditioned medium from 24-48 h transfected BOSC-23 cultures, in the presence of 40 μg/ml polybrene. One day later the medium was replaced with fresh virus-containing medium. Cells were trypsinized, split 1:2 into HC11 growth medium containing 0.75 μg/ml puromycin and selected for 3-4 days. One day before harvesting, the cells were cultured in the absence of puromycin, trypsinized cells were washed, resuspended at 0.5-1.0×10⁶ cells/10 μl PBS and implanted in the fat pad as described in below.

### Example 2

Mammary Gland Transplantation

All experiments were performed with Balb/c mice. Transplants were performed on 8-10 week-old females. The anesthetized mouse was pinned on a cork board and scrubbed with 70% ethanol. Mice were shaved in the region of the left third mammary gland. A small incision of 5 mm length was made 1-2 mm horizontally above the spinal end of the third mammary gland. 0.5-1.0×10⁶ cells were injected using an insulin or Hamilton syringe with a 30 G needle directly into the intact mammary gland fat pad.

The injection of mammary cells into the mammary gland represents a means for introducing cells into the orthotopic site without the removal of host tissue prior to the introduction of the cells.

### Example 3

Transplanted HC-11 Cells Form Mammary Tumors

Whole mount analysis was performed on selected mammary glands. Glands were wholemounted, fixed a minimum of 2 days in Carnoy's fixative (ethanol:chloroform:glacial acetic acid (6:I:I)), defatted in ethanol 2 days, rehydrated and stained with Carmine Red. Tumor material was fixed in formaldehyde containing 1% acetic acid and embedded for haematoxylin and eosin staining.

HCll-puro control cells only produced small areas of growth. Implanted HCII-NeuT cells produced tumors, while no tumors were detected in mice injected with HCll-puro control cells. The histology of the tumors is similar to what has been observed in the MMTV-LTR NeuT transgenic strain as described in Cardiff and Wellings (1999) J. Mamm. Gland Biol. Neoplasia 4: 105-122.

This data confirmed the ability of the neuT gene to act as an oncogene and regulate tumor development. Other potentially oncogenic nucleic acids can be identified in a similar manner.

### Example 4

In Vivo Efficacy Studies Using EGFR-KI and Taxol

Tumors developed after a latency period of four to six weeks. The mice were thereafter treated daily orally over a 19 or 22 day period with 0, 38, 75, 2×75 mg/kg body weight EGFR-kinase inhibitor (N;7-9 per group) or intravenously with 10 mg/kg body weight Taxol.

Oral Treatment with EGFR-KI in this transgenic organ model showed clear anti-tumor efficacy in a dose-dependent manner in the range between 38 and 75 mg/kg body weight. The anti-proliferative effect appeared to be minimally increased at 75 mg/kg/day twice per day. In contrast, treatment with Taxol showed no significant reduction of tumor growth or cell proliferation in this model.

This data identified that EGFR-kinase inhibitors are potential anti-tumor agents.

Finally, it will be appreciated that various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the fields of cell biology, molecular biology or related fields are intended to be within the scope of the present invention.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A method of forming an orthotopic solid tumor in a host, the method comprising the steps of introducing cells into an orthotopic site in the host; and allowing the introduced cells to form a tumor, the cells being transformed by introduction of exogenous nucleic acid into the cells, wherein host tissue is not removed from the orthotopic site prior to the step of introducing the cells into the orthotopic site.

2. A method according to claim 1, wherein the exogenous nucleic acid comprises an oncogene or a proto-oncogene.

3. A method according to claim 2, wherein the oncogene or proto-oncogene comprises an activated oncogene.

4. A method according to claim 2, wherein the oncogene or proto-oncogene comprises a tyrosine kinase receptor oncogene or proto-oncogene.

5. A method according to claim 4, wherein the tyrosine kinase receptor is a member of the epidermal growth factor receptor family.

6. A method according to claim 5, wherein the tyrosine kinase receptor is erbB2.

7. A method according to claim 1, wherein the cells are transformed by the introduction of exogenous nucleic acid prior to the step of introducing the cells into the orthotopic site.

8. A method according to claim 1, wherein 5×10⁵ to 3×10⁷ cells are introduced into the orthotopic site in the host.

9. A method according to claim 1, wherein the cells are introduced into the orthotopic site by inoculation of cells into the orthotopic site.

10. A method according to claim 1, wherein the host is a rodent.

11. An animal comprising an orthotopic tumor produced according to the method of claim 1.

12. A method of identifying an anti-tumor agent, the method comprising the steps of:
(a) producing an orthotopic solid tumor in a host by (i) introducing cells into an orthotopic site in the host and (ii) allowing the cells to form a tumor, the cells being transformed by introduction of exogenous nucleic acid into the cells, wherein host tissue is not removed from the orthotopic site prior to the step of introducing the cells into the orthotopic site;
(b) administering an amount of a test compound to the host; and
(c) identifying an ability of the test compound to inhibit development of the tumor, and therefrom identifying an anti-tumor agent.

13. A method according to claim 12, wherein the exogenous nucleic acid comprises an oncogene or a proto-oncogene.

14. A method according to claim 13, wherein the oncogene or proto-oncogene comprises an activated oncogene.

15. A method according to claim 12, wherein the oncogene or proto-oncogene comprises a tyrosine kinase receptor oncogene or proto-oncogene.

16. A method according to claim 14, wherein the tyrosine kinase receptor is a member of the epidermal growth factor receptor family.

17. A method according to claim 16, wherein the tyrosine kinase receptor is erbB2.

18. A method according to claim 12, wherein the cells are transformed by the introduction of exogenous nucleic acid prior to the step of introducing the cells into the orthotopic site.

19. A method according to claim 12, wherein 5×10⁵ to 3×10⁷ cells are introduced into the orthotopic site in the host.

20. A method according to claim 12, wherein the cells are introduced into the orthotopic site by inoculation of cells into the orthotopic site.

21. A method according to claim 12, wherein the host is a rodent.

22. An anti-tumor agent identified according to the method of claim 12.

23. A method of forming a mammary tumor in an animal, the method comprising the steps of introducing transformed mammary or mammary-derived cells into a site in a mammary gland in the animal; and allowing the introduced cells to form a tumor, the cells being transformed by introduction of an oncogene or proto-oncogene into the cells, wherein animal tissue is not removed from the site prior to the step of introducing the transformed cells.

24. An animal comprising a tumor produced according to the method of claim 23.
